(19) United States
(12) Patent Application Publication
(10) Pub. No.: US 2026/0351387 A1
(43) Pub. Date: Nov. 5, 2026

(54) METHOD AND SYSTEM TO DETERMINE NEED FOR HOSPITAL ADMISSION AFTER ELECTIVE SURGICAL PROCEDURES

(71) Applicant: NEW YORK SOCIETY FOR THE RELIEF OF THE RUPTURED AND CRIPPLED, MAINTAINING THE HOSPITAL FOR SPECIAL, New York, NY (US)

(72) Inventors: Tony S. Shen, New York, NY (US); Matthew Wickersham, New York, NY (US); Nicholas Bartelo, New York, NY (US)

(21) Appl. No.: 19/102,011

(22) PCT Filed: Aug. 7, 2023
(86) PCT No.: PCT/US2023/029610

§ 371 Date: Feb. 7, 2025

Related U.S. Application Data
(60) Provisional application No. 63/396,101, filed on Aug. 8, 2022.
(60) Provisional application No. 63/478,252, filed on Jan. 3, 2023.

(51) Int. Cl. G16H 40/20; G16H 10/60; G16H 50/30; G16H 50/70

(52) U.S. Cl. CPC G16H 40/20; G16H 10/60; G16H 50/30; G16H 50/70

(57) ABSTRACT

A computer-implemented method includes: accessing electronic healthcare records of a group of patients, wherein each patient has received an elective surgical procedure; extracting a data structure encoding a plurality of features of each patient from the group of patients, wherein a subset of the group of patients undergo at least one hospital-based intervention after receiving the elective surgical procedure; determining, using a machine learning algorithm that operates on the data structure, a Shapley value for each of the features, wherein the Shapley value indicates a likelihood for each patient with a corresponding feature to receive at least one hospital-based intervention; identifying a subset of the plurality of features; and based on the identified subset of features, establishing a predictive tool to predict a combined likelihood for a patient to receive a hospital-based intervention.

### CROSS REFERENCE TO RELATED APPLICATIONS

This application claims the priority benefit of U.S. Provisional Application No.

63/396,101, filed Aug. 8, 2022, and U.S. Provisional Application No. 63/478,252, filed Jan. 3, 2023, the content of each of which is incorporated by reference in its entirety herein.

### TECHNICAL FIELD

This description generally relates to treatment and management of elective surgical procedure, for example, those for degenerative conditions such as osteoarthritis (OA) in humans.

### BACKGROUND

Osteoarthritis (OA) is an increasingly common and debilitating condition worldwide, afflicting more than 50 million individuals in the United States and over 500 million globally. This degenerative condition frequently involves the knee and/or the hip, carrying a substantial burden for patients given the pain, disability, decreased quality of life, and rise in mortality, particularly from cardiovascular incidents. Both total hip arthroplasty (THA) and total knee arthroplasty (TKA) deliver excellent outcomes for end-stage OA, reliably providing substantial pain relief and improved quality of life in most patients. However, both procedures still confer increased healthcare costs related to hospital charges, implant costs, physician fees, and post-operative care. In particular, some patients who have received a THA or TKA procedure will require a subsequent hospital-based intervention (HBI), which can add to the cost significantly. Thus, one problem confronting healthcare professionals and institutions is determining prior to surgery which patients are likely to require a HBI after surgery and which can be discharged from hospital care. As used herein HBI refers to post-surgical procedures and treatments such as for example blood transfusions, supplemental or corrective surgery, administration of intravenous medications, urinary catheterization that are to be performed in a hospital setting. By way of illustration, many patients after an initial total joint arthroplasty will need a revision of the primary THA or TKA. From 2014 to 2030, revision THA and TKA incidences are projected to increase by 70% and 182%, respectively. Beyond revision surgeries, patients may need an additional total joint arthroplasty later in life. There is up to a 45% chance of needing a THA or TKA in a contralateral cognate joint and up to a 5% chance in a noncognate joint within 20 years of the primary THA or TKA. Given these growing and high rates of subsequent hip or knee surgeries, the availability of an objective computerized solution to predict whether someone will have an HBI in these populations can greatly improve the quality of machine-implemented predictions and guide healthcare works to navigate complex cases a more targeted manner and with enhanced precision. Thus, there is a need for computerized solution to predict the need for an HBI after an elective surgical procedure such as a THA or a TKA.

### SUMMARY

In one aspect, some implementations of the present disclosure provide a computer-implemented method for determining the likelihood that a patient will require a HBI after undergoing a surgical procedure such as a TKA or THA. The method includes the steps of: accessing a database comprising electronic healthcare records of a group of patients, wherein each patient has received an elective surgical procedure; based on the electronic healthcare records, extracting a data structure encoding a plurality of features of each patient from the group of patients, wherein a subset of the group of patients have received at least one hospital-based intervention after receiving the elective surgical procedure; determining, using a machine learning algorithm that operates on the data structure, a Shapley value for each of the plurality of features, wherein the Shapley value indicates a likelihood for each patient with a corresponding feature to receive at least one hospital-based intervention; identifying a subset of the plurality of features with absolute Shapley values higher than those of remaining features of the plurality of features; and predicting, based on a sum of the Shapley values for the identified subset of features, a combined likelihood for the new patient to receive a hospital-based intervention.

Implementations may include one or more of the following features.

Establishing the predictive tool may include: summing the Shapley values of the identified subset of features; and stratifying the summed SHAP values into a plurality of bins corresponding to respective levels of risk.

The method may further include: splitting the electronic healthcare records of the group of patients into a testing subgroup, and a validating subgroup, wherein the electronic healthcare records of the testing subgroup are used to establish the predictive tool. The method may further include: validating the predictive model using the electronic healthcare records of the validating subgroup of patient; and at least based on results of validating the predictive model, iteratively refining the predictive model.

The features may include: a preoperative factor, a demographic factor, and a perioperative factor, and wherein the elective surgical procedure comprises at least one of: a hip arthroplasty, and a knee arthroplasty.

The machine learning algorithm may include: a logistic regression (LR) algorithm, a linear discriminant analysis (LDA) algorithm, a Gaussian Naïve Bayes (GNB) algorithm, a random forest (RF) algorithm, an extreme Gradient Boosting (XGBoost) algorithm, a multilayer perceptron (MLP) algorithm, and an extra tress (ET) algorithm. The method may further include: benchmarking the machine learning algorithm by at least one metric, wherein the at least one metric comprises: an area under receiver operating curve (AUROC), an area under precision-recall curve (AUPRC), an accuracy, a precision, a sensitivity, and a specificity.

In another aspect, some implementations of the present disclosure provide a computer system comprising at least one hardware processor and at least one display, wherein the at least one hardware processor is configured to perform operations of: accessing a database comprising electronic healthcare records of a group of patients, wherein each patient has received an elective surgical procedure; based on the electronic healthcare records, extracting a data structure encoding a plurality of features of each patient from the group of patients, wherein a subset of the group of patients have received at least one hospital-based intervention after receiving the elective surgical procedure; determining, using a machine learning algorithm that operates on the data structure, a Shapley value for each of the plurality of features, wherein the Shapley value indicates a likelihood for each patient with a corresponding feature to receive at least one hospital-based intervention; identifying a subset of the plurality of features with absolute Shapley values higher than those of remaining features of the plurality of features; and predicting, based on a sum of the Shapley values for the identified subset of features, a combined likelihood for the new patient to receive a hospital-based intervention.

Implementations of the present disclosure may include one or more of the following features.

Establishing the predictive tool may include: summing the Shapley values of the identified subset of features; and stratifying the summed SHAP values into a plurality of bins corresponding to respective levels of risk.

The operations may further include: splitting the electronic healthcare records of the group of patients into a testing subgroup, and a validating subgroup, wherein the electronic healthcare records of the testing subgroup are used to establish the predictive tool. The operations may further include: validating the predictive model using the electronic healthcare records of the validating subgroup of patient; and at least based on results of validating the predictive model, iteratively refining the predictive model.

The features may include: a preoperative factor, a demographic factor, and a perioperative factor, and wherein the elective surgical procedure comprises at least one of: a hip arthroplasty, and a knee arthroplasty.

The machine learning algorithm may include: a logistic regression (LR) algorithm, a linear discriminant analysis (LDA) algorithm, a Gaussian Naïve Bayes (GNB) algorithm, a random forest (RF) algorithm, an extreme Gradient Boosting (XGBoost) algorithm, a multilayer perceptron (MLP) algorithm, and an extra tress (ET) algorithm. The operations may further include: benchmarking the machine learning algorithm by at least one metric, wherein the at least one metric comprises: an area under receiver operating curve (AUROC), an area under precision-recall curve (AUPRC), an accuracy, a precision, a sensitivity, and a specificity.

In yet another aspect, the implementations provide a non-transitory computer-readable medium comprising software instructions that, when executed by one or more computer processors, cause the one or more computer processors to perform operations of: accessing a database comprising electronic healthcare records of a group of patients, wherein each patient has received an elective surgical procedure; based on the electronic healthcare records, extracting a data structure encoding a plurality of features of each patient from the group of patients, wherein a subset of the group of patients have received at least one hospital-based intervention after receiving the elective surgical procedure; determining, using a machine learning algorithm that operates on the data structure, a Shapley value for each of the plurality of features, wherein the Shapley value indicates a likelihood for each patient with a corresponding feature to receive at least one hospital-based intervention; identifying a subset of the plurality of features with absolute Shapley values higher than those of remaining features of the plurality of features; and predicting, based on a sum of the Shapley values for the identified subset of features, a combined likelihood for the new patient to receive a hospital-based intervention.

Implementations may include one or more of the following features.

Establishing the predictive tool may include: summing the Shapley values of the identified subset of features; and stratifying the summed SHAP values into a plurality of bins corresponding to respective levels of risk.

The operations may further include: splitting the electronic healthcare records of the group of patients into a testing subgroup, and a validating subgroup, wherein the electronic healthcare records of the testing subgroup are used to establish the predictive tool. The operations may further include: validating the predictive model using the electronic healthcare records of the validating subgroup of patient; and at least based on results of validating the predictive model, iteratively refining the predictive model.

The features may include: a preoperative factor, a demographic factor, and a perioperative factor, and wherein the elective surgical procedure comprises at least one of: a hip arthroplasty, and a knee arthroplasty.

The machine learning algorithm may include: a logistic regression (LR) algorithm, a linear discriminant analysis (LDA) algorithm, a Gaussian Naïve Bayes (GNB) algorithm, a random forest (RF) algorithm, an extreme Gradient Boosting (XGBoost) algorithm, a multilayer perceptron (MLP) algorithm, and an extra tress (ET) algorithm. The operations may further include: benchmarking the machine learning algorithm by at least one metric, wherein the at least one metric comprises: an area under receiver operating curve (AUROC), an area under precision-recall curve (AUPRC), an accuracy, a precision, a sensitivity, and a specificity.

The details of one or more aspects of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

Embodiments will now be described, by way of example only, with reference to the drawings, in which:

FIGS. 1A-1B show diagrams illustrating an example of a work flow according to some implementations of the present disclosure.

FIG. 2 is a table showing examples of factors that define hospital-based intervention (HBI) in some implementations of the present disclosure.

FIG. 3 is a chart showing a full list of all input features as used by the AI/ML model of some implementations of the present disclosure.

FIG. 4 is a table showing the comparative performance of various models operating on a composite of all input features (from FIG. 3) to predict patients who required an HBI after THA or TKA according to some implementations of the present disclosure.

FIG. 5 shows an example of selecting a subset of input features from the full list of all input features that have contributed the most to the output of the model predictions according to some implementations of the present disclosure.

FIG. 6A is a table showing examples of the comparative performance of various models operating on the selected input features (marked with asterisks in FIG. 3) to identify patients in a testing cohort of patients who required an HBI after THA or TKA according to some implementations of the present disclosure.

FIG. 6B is a table showing examples of the comparative performance of various models operating on the selected input features (marked with asterisks in FIG. 3) to predict patients in a validation cohort of patients who required an HBI after THA or TKA according to some implementations of the present disclosure.

FIG. 7A shows examples of the absolute values of the SHAP feature importance for the selected input features in a cohort of patients according to some implementations of the present disclosure.

FIG. 7B shows examples of the SHAP feature importance values for the selected input features in an individual patient according to some implementations of the present disclosure.

FIG. 7C shows an example of comparing and contrasting the histogram of summed up SHAP values for patients in two distinct groups, as identified by some implementations of the present disclosure.

FIG. 8A shows examples of the percentages of HBIs in all risk categories for two cohort of patients according to some implementations of the present disclosure.

FIGS. 8B and 8C compare the prediction performance of the example of FIG. 8A with known prior art.

FIGS. 9A to 9B show examples of a user interface on a risk tool according to some implementations of the present disclosure.

FIG. 10 shows an example of a computer system used by some implementations of the present disclosure.

Like reference symbols in the various drawings indicate like elements.

### DETAILED DESCRIPTION

Current patient selection guidelines for ambulatory hip or knee arthroplasty are largely based on anecdotal data and vary from institution to institution. Current practice is without an objective and data-driven strategy to determine the safest disposition (discharge strategy/planning) for patients undergoing the replacement procedure. Implementations of the system described in the present disclosure address this problem in hip and knee arthroplasty using a combination of machine learning models and a risk stratification instrument. For example, the implementations may build a machine learning model that identifies preoperative and perioperative factors associated with HBI in a large cohort of patients (e.g., tens of thousands, if not more). The model can be developed by collecting data from medical records that capture, in the large cohort of patients, the occurrence of an HBI as well as a large number (e.g., 64 or more) other preoperative and perioperative features. The implementations may apply multiple supervised machine learning models to this data to generate a predictive model. i.e. a model that will predict the likelihood that the patient will require a post-surgical HBI. Alternative statistical methods may also be used to build this predictive model that do not involve supervised machine learning.

In this predictive model, the implementations may operate a number of risk stratification applications based on, for example, SHAP values (Shapley Additive explanations) capable of dynamically assessing the contribution of each feature relative to each other. A Shapley value is the average marginal contribution of an instance of a feature among all possible coalitions. Shapley Additive Explanations (SHAP), was introduced by Lundberg and Lee in 2017 (“A Unified Approach to Interpreting Model Predictions” 31st Conference on Neural Information Processing Systems (NIPS 2017), Long Beach, CA, USA) and is a technique for interpreting the predictions of Machine Learning models using a summation of the Shapely values for respective input features. The risk stratification applications may incorporate only preoperative variables and demographic factors captured during routine preoperative evaluations. The risk stratification applications may also incorporate perioperative variables available during or soon after surgery. The risk stratification applications may be used to predict, e.g., the need for hospital-based intervention (HBI), as well as hospital admission and length of hospital stay. For example, if a patient requires a HBI after replacement surgery, which includes blood transfusions, urinary catheterization, intravenous medication, and other procedures, then hospital admission is the most appropriate disposition for this patient after surgery. If a patient does not require an HBI, then discharge home after surgery is safe and appropriate. Alternative strategies could also have been used to build risk stratification instruments.

The implementations of the present disclosure have several advantages over existing technology. For example, the implementations are based on models driven by objective data from a large population of patients. The models can be further improved as more data is collected. Moreover, the implementations can generate a customized risk profile for each individual patient based on patient-specific characteristics. The implementations thus incorporate a data-centric approach, rather than relying on anecdotal evidence as used in current practice. The implementations can be generalized to any elective surgical procedure for which inpatient data is available from each patient's postoperative admission. Such elective surgeries can include shoulder arthroplasty, single level spinal fusions, and anterior cervical discectomy and fusion, among many others.

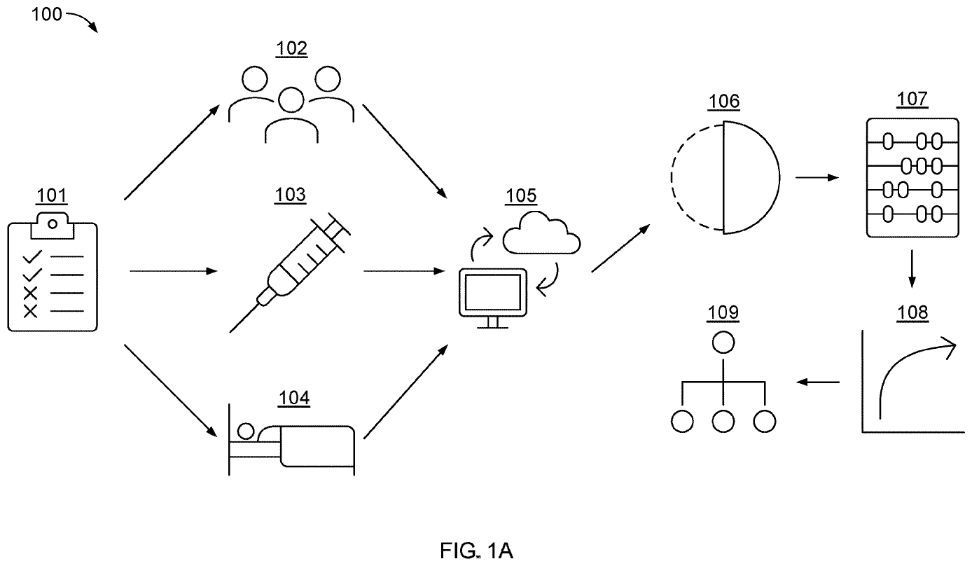

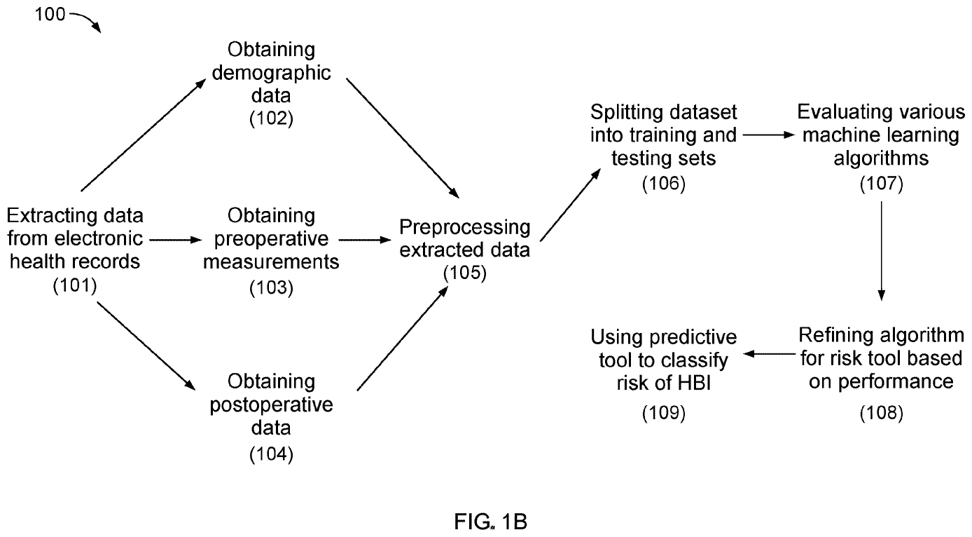

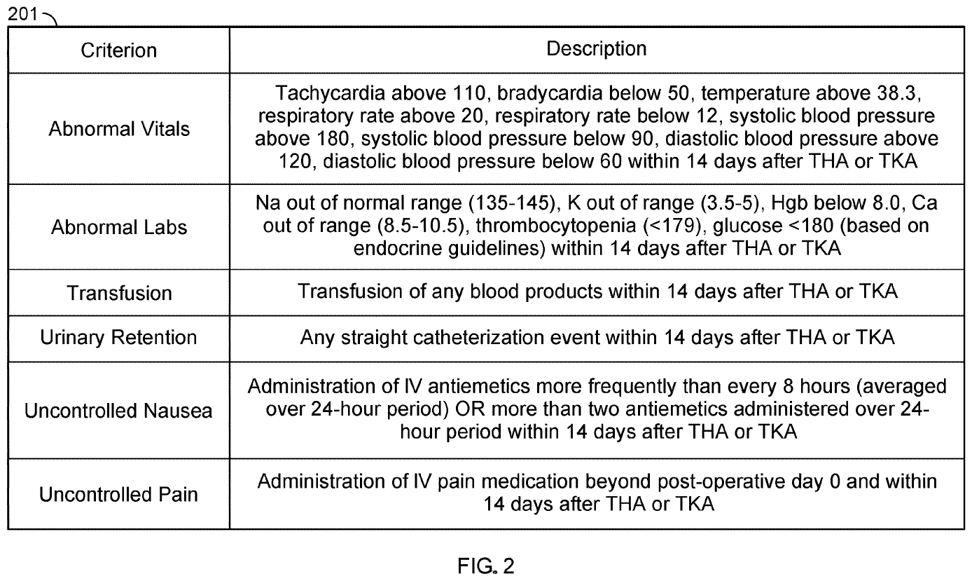

FIGS. 1A-1B show diagrams illustrating an example of a process 100 according to some implementations of the present disclosure. As illustrated, data from electronic health records may be extracted (101). The electronic health records may be stored in multiple databases where the record of each patient is de-identified (i.e., anonymized). The electronic health records for a given patient undergoing THA or TKA show whether the patient subsequently required a hospital-based intervention (HBI) after surgery. A hospital-based intervention (HBI) can be further defined as having a condition in need of hospital intervention after the THA or TKA surgery. As tabulated in FIG. 2, examples can include any of the following conditions within 14 days during admission after a THA or TKA: abnormal vital, abnormal lab, a transfusion, urinary retention, uncontrolled nausea, and/or uncontrolled pain.

The electronic health records can generally encompass demographic, genomic, questionnaire, office visit, hospital stay, biomarker lab measurement, and imaging data. Such data can be captured from patients at high-volume orthopedic specialty hospitals, or general hospitals with an orthopedic specialty practice. As illustrated, the demographic data can include age, race, sex information, which can be obtained (102). Some implementations may obtain preoperative measurements (103) and postoperative data (104). The implementations may additionally obtain perioperative data during the surgical replacement procedure.

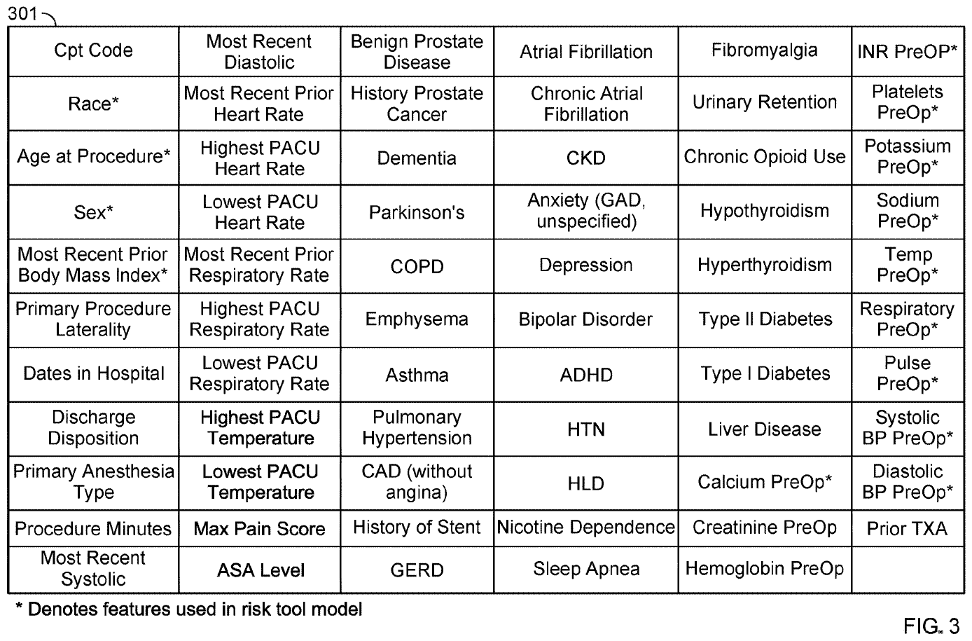

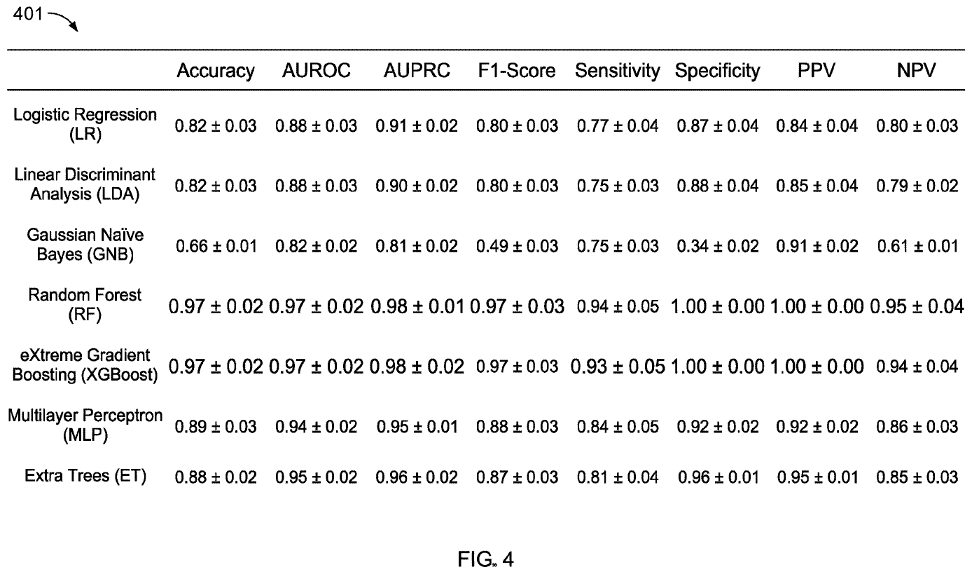

In some implementations, preoperative data can include preoperative vital or lab measurement taken within 90 days prior to the THA or TKA. In the case of multiple vitals or labs, the measurement closest to the date of the THA or TKA can be used. Similarly, postoperative data can include postoperative vital or lab measurement while perioperative data can include such data obtained during the surgical procedure. These examples can be expanded under various guidelines, such as, for example, reporting guidelines known as Transparent Reporting of a Multivariable Prediction Model for Individual Prognosis or Diagnosis (TRIPOD). FIG. 3 shows table 301 that tabulates a total of sixty-five (65) examples of demographic, preoperative, perioperative, and postoperative input features. The implementations demonstrate the use of various models that operate on these input features to predict patients who would subsequently require an HBI after THA or TKA, as further explained in association with FIGS. 4-10.

Returning to FIGS. 1A-1B, after extraction, the pre, peri and post-operative data may be pre-processed, for example, into a structured format for additional processing (105). The implementations may then split the extracted and pre-processed data into a training subset and a testing subset (106). The training subset is also known as the training cohort, in which the data is applied to, for example, supervised machine learning models that predict various health outcomes based on the input features. In particular, the training cohort can be used to develop a risk stratification tool. The implementations may then evaluate various machine learning models and the risk stratification tool using data from the testing subset, also known as the testing cohort (107). Based on the evaluation results, the implementations may then refine the machine learning models, including the selection of a subset of input features, to build and refine the risk stratification tool (108). The implementations may then apply the risk tool to predictively classify the risk of HBI for one or more new patients (109).

Some implementations may use a composite of all input features, for example, all of the 65 demographic, preoperative, perioperative, and postoperative input features of FIG. 3 to predict patients who are more likely to require an HBI after receiving THA or TKA. FIG. 4 shows table 401 that tabulates the comparative merits from some implementations for various machine learning tools including, for example, Logistic Regression (LR), Linear Discriminatory Analysis (LDA), Gaussian Naïve Bayes (GNB), Random Forest (RF), eXtreme Gradient Boosting (XGBoost), Multilayer Perception (MLP), and Extra Trees (ET). As illustrated, RF (Random Forest) and XGBoost (extreme Gradient Boosting) models demonstrate the highest AUROC of 0.97 (95% CI=0.96-0.98), AUPRC of 0.98 (95% CI=0.96-1.00), and F1-Score of 0.97 (95% CI=0.95-1.00). As used in the art, AUROC refers to the Area Under an ROC (Receiver operating characteristic) Curve, while AUPRC refers to the Area Under the Precision-Recall Curve. A Precison Recall Curve (PR curve) is a graph with Precision values on the y-axis and Recall values on the x-axis. The PR curve contains TP/(TP+FN) on the y-axis and TP/(TP+FP) on the x-axis. Moreover, RF and XGBoost models also exhibit the highest performing metrics for specificity, sensitivity, positive predictive value (PPV) and negative predictive value (NPV).

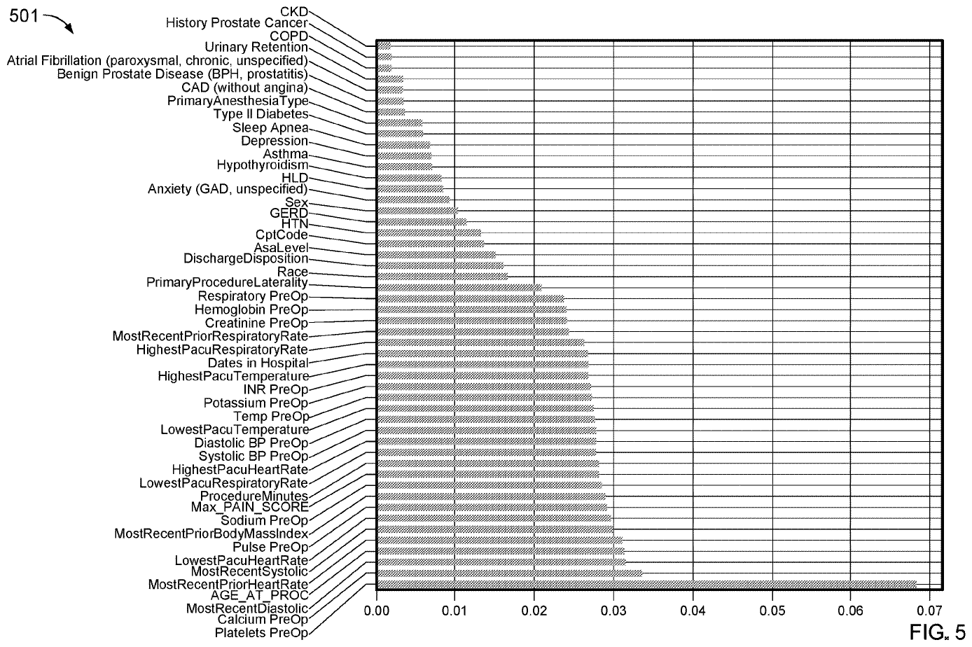

In the example above, while all 65 input features used in the composite model could be used to reassess risk of subsequent hospital-based interventions, assessing a patient's initial risk, e.g., before the THA or TKA replacement procedure, for requiring a later HBI could help determine, by the time of THA/TKA replacement procedure, which patients are suitable for ambulatory procedures. To develop a model based on only demographic and preoperative variables, feature selection may be performed using the RF model to identify the preoperative and demographic features that are the most contributive to the composite model. FIG. 5 shows chart 501 illustrating the ranking of the preoperative and demographic input features in the composite model, as measured by a magnitude of correlation with the outcome, i.e., whether or not a later HBI would be needed. The following table summarizes acronyms used in chart 501.
CKD—chronic kidney disease
COPD—chronic obstructive pulmonary disease
CAD—coronary artery disease
HLD—hyperlipidemia
GERD—gastroesophageal reflux disease
HTN—hypertension

Using the same cohort from the composite model, implementations may derive a risk tool using the same training subset and testing subset with, for example, a subset of input features selected based on the top ranking input features.

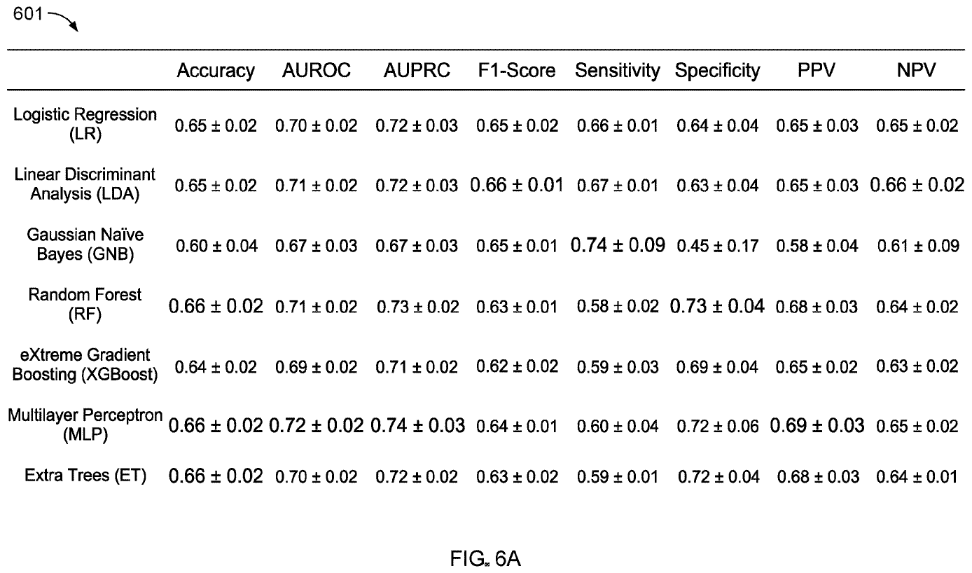

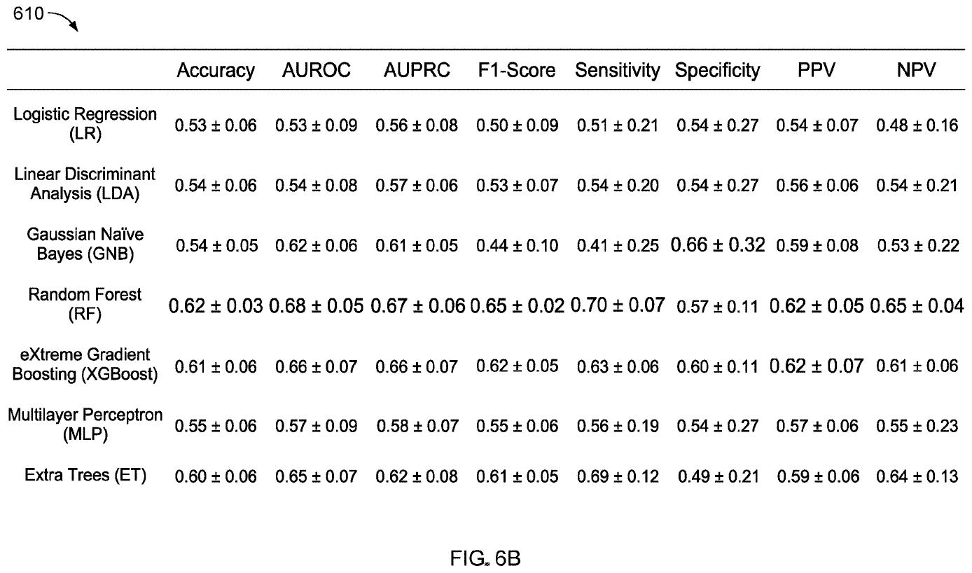

Using the selected input features, the same supervised machine learning models can be applied to the testing subset (where the outcome (whether a HBI is needed) is treated as known during derivation of the model parameters) to generate the figure of merits for the respective machine learning models, as shown in table 601 of FIG. 6A. As illustrated, the Multilayer Perception (MLP) model has the highest AUROC of 0.72 (95% CI=0.70-0.74) and AUPRC of 0.74 (95% CI=0.71-0.77), while Linear Discriminant Analysis (LDA) has the highest F1-Score of 0.66 (95% CI=0.65-0.67).

When the various models are applied to the testing subset whose data records are not used to build the ML models of FIG. 6A, the results are shown in FIG. 6B. For this subset, the outcome (whether a HBI is needed) is treated as unknown, and the model prediction is compared with the actual record to determine validity. The comparative merits are presented in table 610 of FIG. 6B. As illustrated, the sensitivity and specificity for all models have dropped (when compared with the testing subset from FIG. 6A). Still, Random Forest (RF) exhibits the highest AUROC of 0.68 (95% CI=0.63-0.73), AUPRC of 0.67 (95% CI=0.61-0.73) and F1-Score of 0.65 (95% CI=0.63-0.67). Additionally, RF has the highest accuracy, sensitivity, PPV, and NPV.

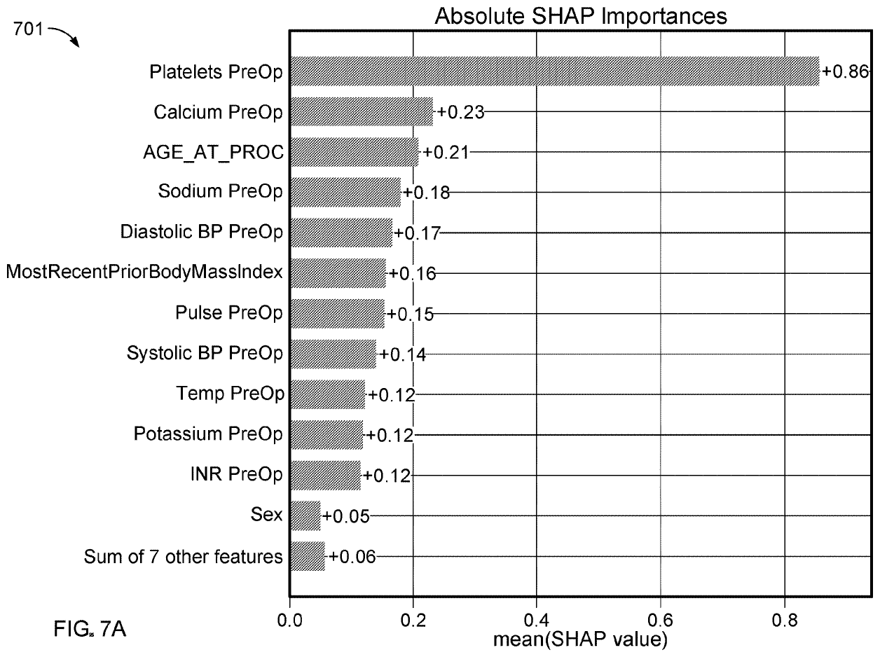

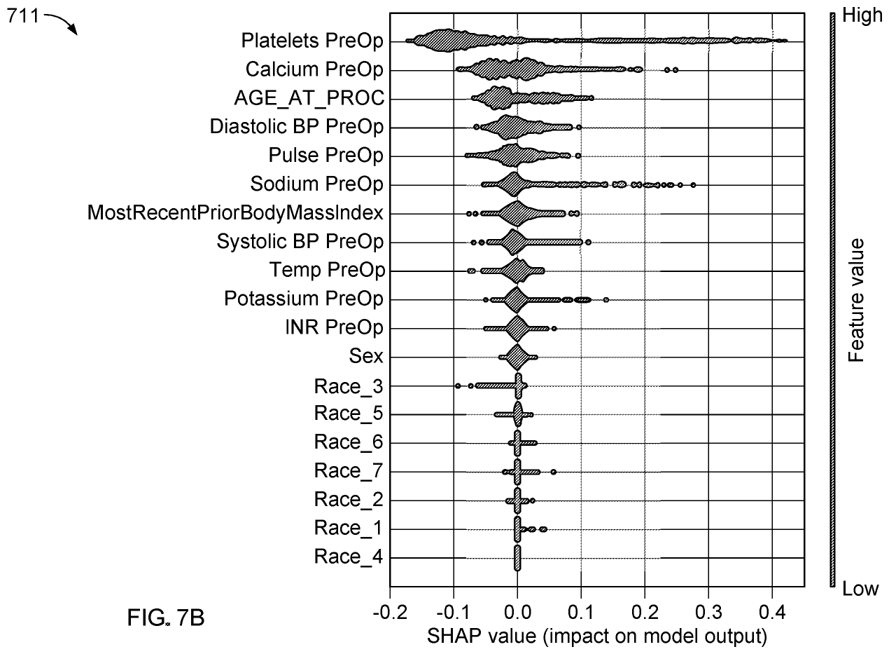

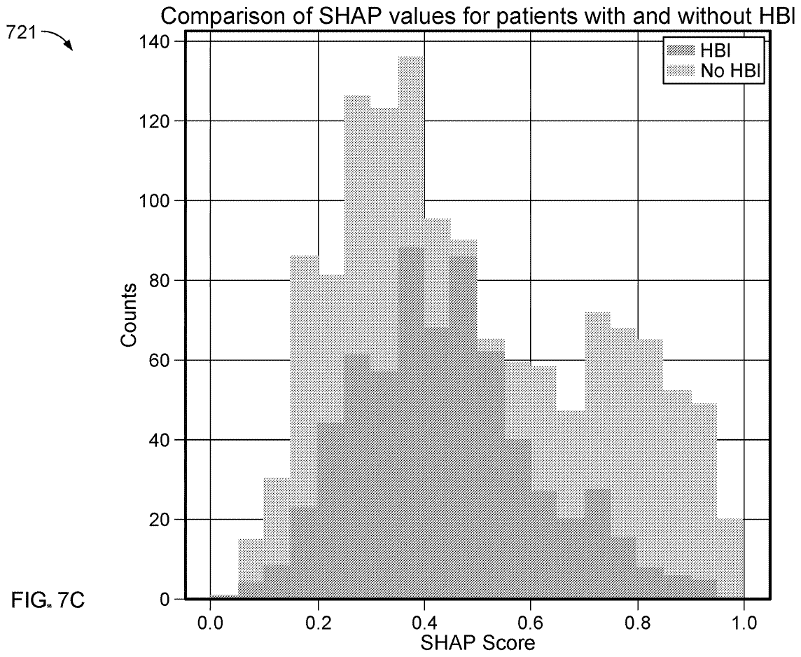

Some implementations may use RF as the model for the risk tool because RF may deliver the best performance metrics. In these implementations, once the RF model has been applied to the input features, Shapely values used by the SHAP additions can be computed for each feature. As illustrated in FIG. 7A, absolute value of Shapley feature importance reveals that preoperative platelet levels, preoperative calcium levels, and a patient's age can have the overall largest impact on the total SHAP score for a given patient. Some implementations may thus select the subset of 13 input features (e.g., preoperative and demographic factors) based on data from the training cohort of patients, as marked by asterisks of FIG. 3. Examples of selected input features can include: level of platelets, level of calcium, diastolic blood pressure, age, pulse rate, systolic blood pressure, BMI, and level of sodium. To build a tool that is useful during the preoperative period, these selected input features are based on information available after a preoperative evaluation. Notably, this Shapley value for each feature could be negative or positive based on the inputs for a given patient, as illustrated in FIG. 7B. After a Shapley value is determined for each feature of a patient, the Shapley values can be summed to obtain a total SHAP score for that patient. Once the normalized SHAP scores are plotted against one another, the SHAP scores for the testing cohort show that patients without an HBI typically have a lower SHAP score than those with an HBI. Tellingly, as revealed in FIG. 7C, the SHAP scores exhibit a bimodal distribution between the patients who required an HBI, and those without an HBI. In other words, the distribution of SHAP scores for patients without HBI and patients with HBI are clustered at different locations. The bimodal distribution highlights the improved statistical precision of the implementations to distinguish two groups, namely, a first group with an HBI, and a second group without an HBI. Significantly, the computerized implementations are driven by objective data, which means the solution can be further improved or refined with additional data (as the cohort of patients grow in size). Thus, the implementations epitomize an improved capability of a computerized solution to statistically differentiate, with high fidelity, two groups of patients. This improved capability adds significantly more to the computer implementation at least in that it provides an unprecedented and objective tool to predict, by leveraging electronic health records that are accumulating daily, and using growing power of Shapley statistics, the post-treatment course of the patient. To date, none of the prior solutions are capable of such statistical differentiation.

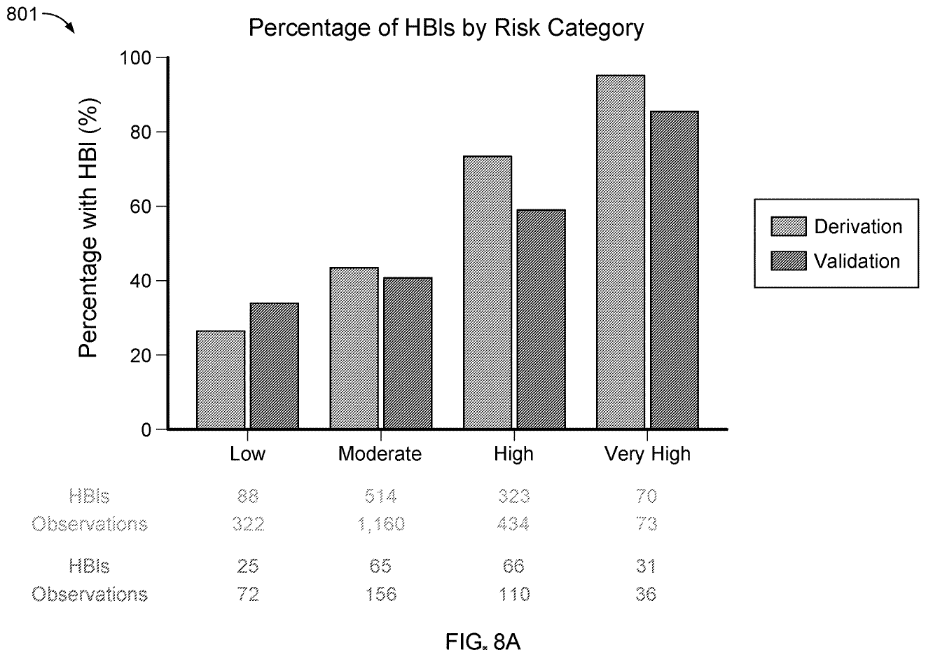

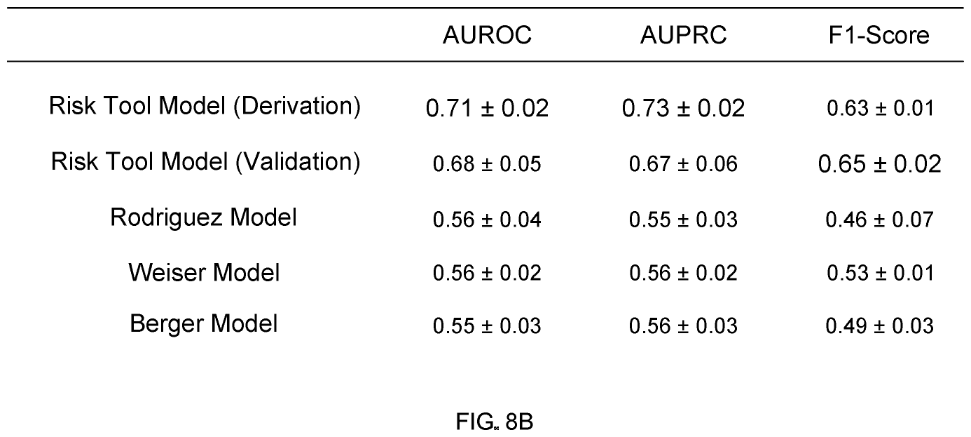

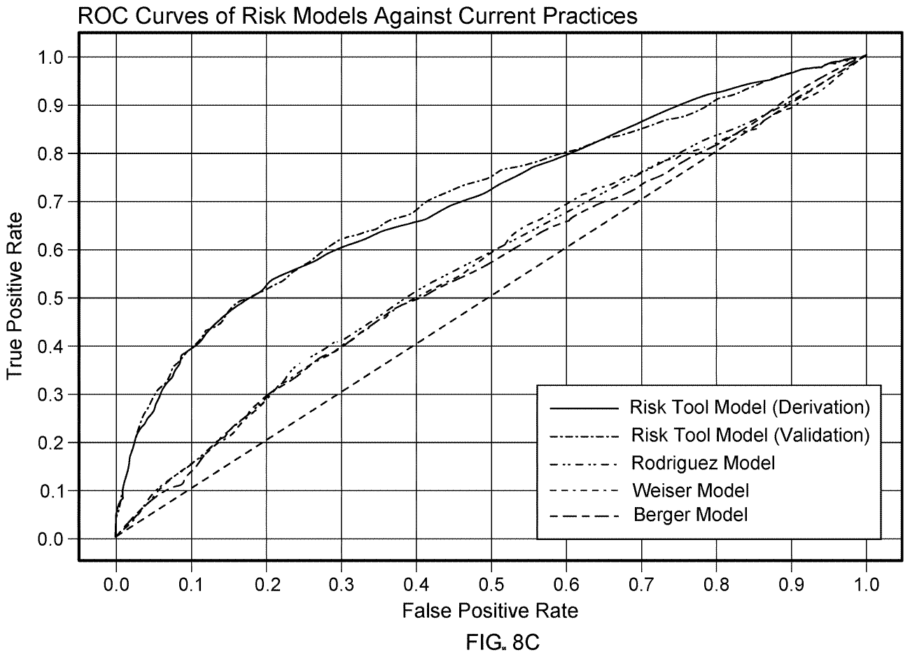

To derive a risk tool based on the relative risk of a patient having an HBI after THA or TKA, implementations may create a number of bins (or levels) based on the normalized SHAP values. In one example, four bins can be created, namely, low risk (0.00-0.20), moderate risk (0.20-0.55), high risk (0.55-0.75), and very high risk (0.75-1.00). These bins can be assessed from data records from both the testing cohort of a first patients' database (labelled as the “derivation” cohort) and the testing cohort of a second patients' database (labelled as the “validation” cohort). The second patient's database is different from the first patient's database, As revealed in FIG. 8A, in both the derivation and validation cohorts, there is an increasing percentage of HBIs as the risk category becomes more severe. Namely, of those patients in the very high risk bin, 95.9% of patients in the derivation cohort had an HBI, and 86.1% of patients in validation cohort had an HBI. The relatively minor percentage drop in the validation cohort demonstrates the feasibility of the implementations to operate on electronic health records not used in model derivation to provide useful insight. Indeed, the results demonstrate the improved statistical power of the underlying computerized tool to differentiate, for example, by the time of replacement surgery, patients in need of a follow-up hospital-based intervention and patients who can be discharged right away. At least for this reason, the implementations provide a specific technique for improving the computation power (e.g., the statistical power to resolve two groups otherwise indistinguishable) of a computerized solution.

Indeed, the superiority of the implementations of the present disclosure is demonstrated by comparisons with known guidelines. For example, the ROC (Receiver operating characteristic) Curve of the model described above has better predictive performance than the Rodriguez, Weiser, and Berger models based on existing published institutional guidelines for same-day discharge after hip or knee replacement. As shown in FIG. 8B, the AUROC, AUPRC, and F1-score metrics are consistently higher when the model described is used. Additionally, as shown in FIG. 8C, when operating at the same the false positive rate, the model described above consistently delivers a higher true positive rate than models based on current practice. Similarly, when operating at the same true positive rate, the model described above consistently delivers a lower false negative rate than models based on current practice. In other words, the implementations of the present disclosure generate predictions with improved statistical precision than current practice.

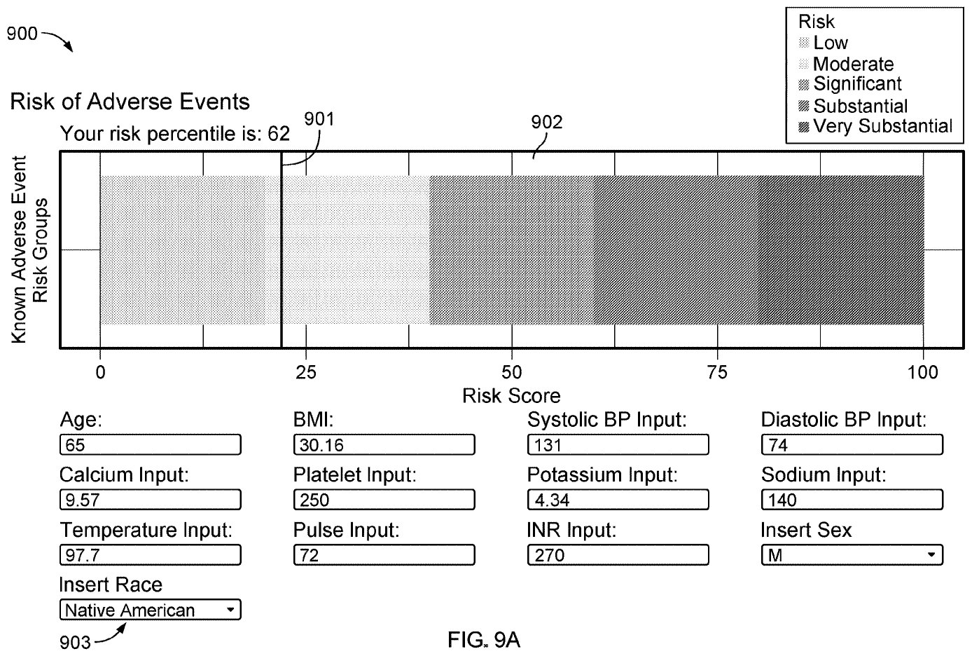

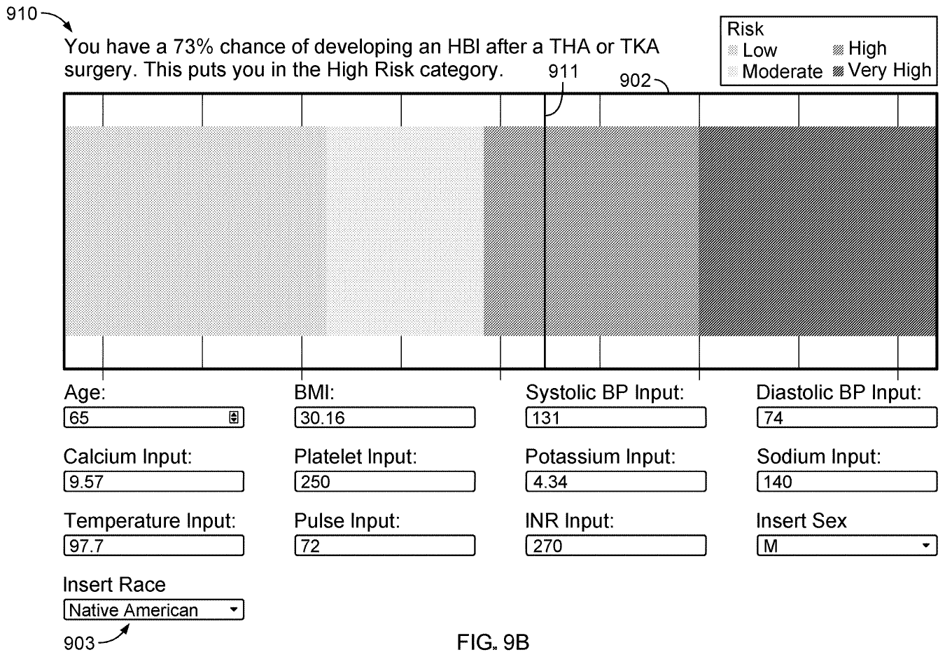

FIGS. 9A and 9B show examples 900 and 910 of a user interface on a risk tool computing device according to some implementations of the present disclosure. The computing device can include a desktop computer, or a mobile computing device. The computing device can also incorporate a client-server architecture so that the input can be collected from a user terminal, and then relayed to a server where the analysis is performed. The client-server configuration may facilitate access to databases whose underlying data is evolving on a day-by-day basis. As illustrated, each user interface may include a number of panels (903) to collect input features. Once the input features are submitted, the risk tool computing device can compute the risk category and then generate a display (902) to the user. The results can be anywhere on the spectrum, from a low-risk determination (e.g., bar 901 in FIG. 9A) to a high-risk determination (e.g., bar 911 FIG. 9B). The implementations of the present disclosure outperform known algorithm in terms of, for example, specificity of the stratification outcome.

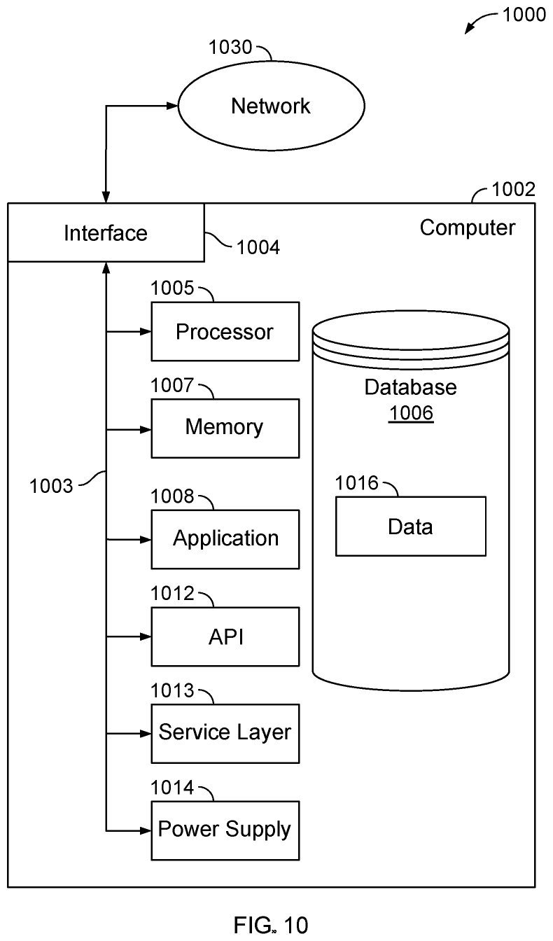

FIG. 10 is a block diagram 1000 illustrating an example of a computer system 1000 used to provide computational functionalities associated with described algorithms, methods, functions, processes, flows, and procedures, according to an implementation of the present disclosure. The illustrated computer 1002 is intended to encompass any computing device such as a server, desktop computer, laptop/notebook computer, wireless data port, smart phone, personal data assistant (PDA), tablet computing device, one or more processors within these devices, another computing device, or a combination of computing devices, including physical or virtual instances of the computing device, or a combination of physical or virtual instances of the computing device. Additionally, the computer 1002 can comprise a computer that includes an input device, such as a keypad, keyboard, touch screen, another input device, or a combination of input devices that can accept user information, and an output device that conveys information associated with the operation of the computer 1002, including digital data, visual, audio, another type of information, or a combination of types of information, on a graphical-type user interface (UI) (or GUI) or other UI. Examples of the UI are demonstrated in FIGS. 9A and 9B.

The computer 1002 can serve in a role in a computer system as a client, network component, a server, a database or another persistency, another role, or a combination of roles for performing the subject matter described in the present disclosure. The illustrated computer 1002 is communicably coupled with a network 1030. In some implementations, one or more components of the computer 1002 can be configured to operate within an environment, including cloud-computing-based, local, global, another environment, or a combination of environments.

The computer 1002 is an electronic computing device operable to receive, transmit, process, store, or manage data and information associated with the described subject matter. According to some implementations, the computer 1002 can also include or be communicably coupled with a server, including an application server, e-mail server, web server, caching server, streaming data server, another server, or a combination of servers.

The computer 1002 can receive requests over network 1030 (for example, from a client software application executing on another computer 1002) and respond to the received requests by processing the received requests using a software application or a combination of software applications. In addition, requests can also be sent to the computer 1002 from internal users, external or third-parties, or other entities, individuals, systems, or computers.

Each of the components of the computer 1002 can communicate using a system bus 1003. In some implementations, any or all of the components of the computer 1002, including hardware, software, or a combination of hardware and software, can interface over the system bus 1003 using an application programming interface (API) 1012, a service layer 1013, or a combination of the API 1012 and service layer 1013. The API 1012 can include specifications for routines, data structures, and object classes. The API 1012 can be either computer-language independent or dependent and refer to a complete interface, a single function, or even a set of APIs. The service layer 1013 provides software services to the computer 1002 or other components (whether illustrated or not) that are communicably coupled to the computer 1002. The functionality of the computer 1002 can be accessible for all service consumers using this service layer. Software services, such as those provided by the service layer 1013, provide reusable, defined functionalities through a defined interface. For example, the interface can be software written in JAVA, C++, another computing language, or a combination of computing languages providing data in extensible markup language (XML) format, another format, or a combination of formats. While illustrated as an integrated component of the computer 1002, alternative implementations can illustrate the API 1012 or the service layer 1013 as stand-alone components in relation to other components of the computer 1002 or other components (whether illustrated or not) that are communicably coupled to the computer 1002. Moreover, any or all parts of the API 1012 or the service layer 1013 can be implemented as a child or a sub-module of another software module, enterprise application, or hardware module without departing from the scope of the present disclosure.

The computer 1002 includes an interface 1004. Although illustrated as a single interface 1004 in FIG. 10, two or more interfaces 1004 can be used according to particular needs, desires, or particular implementations of the computer 1002. The interface 1004 is used by the computer 1002 for communicating with another computing system (whether illustrated or not) that is communicatively linked to the network 1030 in a distributed environment. Generally, the interface 1004 is operable to communicate with the network 1030 and comprises logic encoded in software, hardware, or a combination of software and hardware. More specifically, the interface 1004 can comprise software supporting one or more communication protocols associated with communications such that the network 1030 or interface's hardware is operable to communicate physical signals within and outside of the illustrated computer 1002.

The computer 1002 includes a processor 1005. Although illustrated as a single processor 1005 in FIG. 10, two or more processors can be used according to particular needs, desires, or particular implementations of the computer 1002. Generally, the processor 1005 executes instructions and manipulates data to perform the operations of the computer 1002 and any algorithms, methods, functions, processes, flows, and procedures as described in the present disclosure.

The computer 1002 also includes a database 1006 that can hold data for the computer 1002, another component communicatively linked to the network 1030 (whether illustrated or not), or a combination of the computer 1002 and another component. For example, database 1006 can be an in-memory, conventional, or another type of database storing data consistent with the present disclosure. In some implementations, database 1006 can be a combination of two or more different database types (for example, a hybrid in-memory and conventional database) according to particular needs, desires, or particular implementations of the computer 1002 and the described functionality. Although illustrated as a single database 1006 in FIG. 10, two or more databases of similar or differing types can be used according to particular needs, desires, or particular implementations of the computer 1002 and the described functionality. While database 1006 is illustrated as an integral component of the computer 1002, in alternative implementations, database 1006 can be external to the computer 1002. As illustrated, the database 1006 holds the previously described data 1016 including, for example, demographic data, pre-operative data, perioperative data, and post-operative data as discussed in FIGS. 1A-1B.

The computer 1002 also includes a memory 1007 that can hold data for the computer 1002, another component or components communicatively linked to the network 1030 (whether illustrated or not), or a combination of the computer 1002 and another component. Memory 1007 can store any data consistent with the present disclosure. In some implementations, memory 1007 can be a combination of two or more different types of memory (for example, a combination of semiconductor and magnetic storage) according to particular needs, desires, or particular implementations of the computer 1002 and the described functionality. Although illustrated as a single memory 1007 in FIG. 10, two or more memories 1007 or similar or differing types can be used according to particular needs, desires, or particular implementations of the computer 1002 and the described functionality. While memory 1007 is illustrated as an integral component of the computer 1002, in alternative implementations, memory 1007 can be external to the computer 1002.

The application 1008 is an algorithmic software engine providing functionality according to particular needs, desires, or particular implementations of the computer 1002, particularly with respect to functionality described in the present disclosure. For example, application 1008 can serve as one or more components, modules, or applications. Further, although illustrated as a single application 1008, the application 1008 can be implemented as multiple applications 1008 on the computer 1002. In addition, although illustrated as integral to the computer 1002, in alternative implementations, the application 1008 can be external to the computer 1002.

The computer 1002 can also include a power supply 1014. The power supply 1014 can include a rechargeable or non-rechargeable battery that can be configured to be either user-or non-user-replaceable. In some implementations, the power supply 1014 can include power-conversion or management circuits (including recharging, standby, or another power management functionality). In some implementations, the power-supply 1014 can include a power plug to allow the computer 1002 to be plugged into a wall socket or another power source to, for example, power the computer 1002 or recharge a rechargeable battery.

There can be any number of computers 1002 associated with, or external to, a computer system containing computer 1002, each computer 1002 communicating over network 1030. Further, the term “client,” “user,” or other appropriate terminology can be used interchangeably, as appropriate, without departing from the scope of the present disclosure. Moreover, the present disclosure contemplates that many users can use one computer 1002, or that one user can use multiple computers 1002.

The subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs, that is, one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal, for example, a machine-generated electrical, optical, or electromagnetic signal that is generated to encode information for transmission to a receiver apparatus for execution by a data processing apparatus. The computer-storage medium can be a machine-readable storage device, a machine-readable storage substrate, a random or serial access memory device, or a combination of computer-storage mediums. Configuring one or more computers means that the one or more computers have installed hardware, firmware, or software (or combinations of hardware, firmware, and software) so that when the software is executed by the one or more computers, particular computing operations are performed.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. In one non-limiting example, the terms “about” and “approximately” mean plus or minus 10 percent or less.

The term “real-time,” “real time,” “realtime,” “real (fast) time (RFT),” “near(ly) real-time (NRT),” “quasi real-time,” or similar terms (as understood by one of ordinary skill in the art), means that an action and a response are temporally proximate such that an individual perceives the action and the response occurring substantially simultaneously. For example, the time difference for a response to display (or for an initiation of a display) of data following the individual's action to access the data can be less than 1 millisecond (ms), less than 1 second(s), or less than 5 s. While the requested data need not be displayed (or initiated for display) instantaneously, it is displayed (or initiated for display) without any intentional delay, taking into account processing limitations of a described computing system and time required to, for example, gather, accurately measure, analyze, process, store, or transmit the data.

The terms “data processing apparatus,” “computer,” or “electronic computer device” (or equivalent as understood by one of ordinary skill in the art) refer to data processing hardware and encompass all kinds of apparatus, devices, and machines for processing data, including by way of example, a programmable processor, a computer, or multiple processors or computers. The apparatus can also be, or further include special purpose logic circuitry, for example, a central processing unit (CPU), an FPGA (field programmable gate array), or an ASIC (application-specific integrated circuit). In some implementations, the data processing apparatus or special purpose logic circuitry (or a combination of the data processing apparatus or special purpose logic circuitry) can be hardware-or software-based (or a combination of both hardware-and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with an operating system of some type, for example LINUX, UNIX, WINDOWS, MAC OS, ANDROID, IOS, another operating system, or a combination of operating systems.

A computer program, which can also be referred to or described as a program, software, a software application, a unit, a module, a software module, a script, code, or other component can be written in any form of programming language, including compiled or interpreted languages, or declarative or procedural languages, and it can be deployed in any form, including, for example, as a stand-alone program, module, component, or subroutine, for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files, for example, files that store one or more modules, sub-programs, or portions of code. A computer program can be deployed to be executed on one computer or on multiple computers that are located at one site or distributed across multiple sites and interconnected by a communication network.

While portions of the programs illustrated in the various figures can be illustrated as individual components, such as units or modules, that implement described features and functionality using various objects, methods, or other processes, the programs can instead include a number of sub-units, sub-modules, third-party services, components, libraries, and other components, as appropriate. Conversely, the features and functionality of various components can be combined into single components, as appropriate. Thresholds used to make computational determinations can be statically, dynamically, or both statically and dynamically determined.

Described methods, processes, or logic flows represent one or more examples of functionality consistent with the present disclosure and are not intended to limit the disclosure to the described or illustrated implementations, but to be accorded the widest scope consistent with described principles and features. The described methods, processes, or logic flows can be performed by one or more programmable computers executing one or more computer programs to perform functions by operating on input data and generating output data. The methods, processes, or logic flows can also be performed by, and apparatus can also be implemented as, special purpose logic circuitry, for example, a CPU, an FPGA, or an ASIC.

Computers for the execution of a computer program can be based on general or special purpose microprocessors, both, or another type of CPU. Generally, a CPU will receive instructions and data from and write to a memory. The essential elements of a computer are a CPU, for performing or executing instructions, and one or more memory devices for storing instructions and data. Generally, a computer will also include, or be operatively coupled to, receive data from or transfer data to, or both, one or more mass storage devices for storing data, for example, magnetic, magneto-optical disks, or optical disks. However, a computer need not have such devices. Moreover, a computer can be embedded in another device, for example, a mobile telephone, a personal digital assistant (PDA), a mobile audio or video player, a game console, a global positioning system (GPS) receiver, or a portable memory storage device.

Non-transitory computer-readable media for storing computer program instructions and data can include all forms of media and memory devices, magnetic devices, magneto optical disks, and optical memory device. Memory devices include semiconductor memory devices, for example, random access memory (RAM), read-only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Magnetic devices include, for example, tape, cartridges, cassettes, internal/removable disks. Optical memory devices include, for example, digital video disc (DVD), CD-ROM, DVD+/−R, DVD-RAM, DVD-ROM, HD-DVD, and BLURAY, and other optical memory technologies. The memory can store various objects or data, including caches, classes, frameworks, applications, modules, backup data, jobs, web pages, web page templates, data structures, database tables, repositories storing dynamic information, or other appropriate information including any parameters, variables, algorithms, instructions, rules, constraints, or references. Additionally, the memory can include other appropriate data, such as logs, policies, security or access data, or reporting files. The processor and the memory can be supplemented by, or incorporated in, special purpose logic circuitry.

To provide for interaction with a user, implementations of the subject matter described in this specification can be implemented on a computer having a display device, for example, a CRT (cathode ray tube), LCD (liquid crystal display), LED (Light Emitting Diode), or plasma monitor, for displaying information to the user and a keyboard and a pointing device, for example, a mouse, trackball, or trackpad by which the user can provide input to the computer. Input can also be provided to the computer using a touchscreen, such as a tablet computer surface with pressure sensitivity, a multi-touch screen using capacitive or electric sensing, or another type of touchscreen. Other types of devices can be used to interact with the user. For example, feedback provided to the user can be any form of sensory feedback. Input from the user can be received in any form, including acoustic, speech, or tactile input. In addition, a computer can interact with the user by sending documents to and receiving documents from a client computing device that is used by the user.

The term “graphical user interface,” or “GUI,” can be used in the singular or the plural to describe one or more graphical user interfaces and each of the displays of a particular graphical user interface. Therefore, a GUI can represent any graphical user interface, including but not limited to, a web browser, a touch screen, or a command line interface (CLI) that processes information and efficiently presents the information results to the user. In general, a GUI can include a plurality of user interface (UI) elements, some or all associated with a web browser, such as interactive fields, pull-down lists, and buttons. These and other UI elements can be related to or represent the functions of the web browser.

Implementations of the subject matter described in this specification can be implemented in a computing system that includes a back-end component, for example, as a data server, or that includes a middleware component, for example, an application server, or that includes a front-end component, for example, a client computer having a graphical user interface or a Web browser through which a user can interact with an implementation of the subject matter described in this specification, or any combination of one or more such back-end, middleware, or front-end components. The components of the system can be interconnected by any form or medium of wireline or wireless digital data communication (or a combination of data communication), for example, a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) using, for example, 802.11 a/b/g/n or 802.20 (or a combination of 802.11x and 802.20 or other protocols consistent with the present disclosure), all or a portion of the Internet, another communication network, or a combination of communication networks. The communication network can communicate with, for example, Internet Protocol (IP) packets, Frame Relay frames, Asynchronous Transfer Mode (ATM) cells, voice, video, data, or other information between networks addresses.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of what can be claimed, but rather as descriptions of features that can be specific to particular implementations. Certain features that are described in this specification in the context of separate implementations can also be implemented, in combination, in a single implementation. Conversely, various features that are described in the context of a single implementation can also be implemented in multiple implementations, separately, or in any sub-combination. Moreover, although previously described features can be described as acting in certain combinations and even initially claimed as such, one or more features from a claimed combination can, in some cases, be excised from the combination, and the claimed combination can be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations can be considered optional), to achieve desirable results. In certain circumstances, multitasking or parallel processing (or a combination of multitasking and parallel processing) can be advantageous and performed as deemed appropriate.

Moreover, the separation or integration of various system modules and components in the previously described implementations should not be understood as requiring such separation or integration in all implementations, and it should be understood that the described program components and systems can generally be integrated together in a single software product or packaged into multiple software products.

Furthermore, any claimed implementation is considered to be applicable to at least a computer-implemented method; a non-transitory, computer-readable medium storing computer-readable instructions to perform the computer-implemented method; and a computer system comprising a computer memory interoperably coupled with a hardware processor configured to perform the computer-implemented method or the instructions stored on the non-transitory, computer-readable medium.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

What is claimed is:

1. A computer-implemented method comprising:
accessing a database comprising electronic healthcare records of a group of patients, wherein each patient has received an elective surgical procedure;
based on the electronic healthcare records, extracting a data structure encoding a plurality of features of each patient from the group of patients, wherein a subset of the group of patients have received at least one hospital-based intervention after receiving the elective surgical procedure;
determining, using a machine learning algorithm that operates on the data structure, a Shapley value for each of the plurality of features, wherein the Shapley value indicates a likelihood for each patient with a corresponding feature to receive at least one hospital-based intervention;
identifying a subset of the plurality of features with absolute Shapley values higher than those of remaining features of the plurality of features; and
based on the identified subset of features, establishing a predictive model such that when electronic healthcare records of a new patient are received by the database, the predictive model predicts, based on the Shapley values for the identified subset of features, a combined likelihood for the new patient to receive a hospital-based intervention.

2. The computer-implemented method of claim 1, wherein the predictive model comprises:
summing the Shapley values of the identified subset of features; and
stratifying the summed Shapley values into a plurality of bins corresponding to respective levels of risk.

3. The computer-implemented method of claim 1, further comprising:
splitting the electronic healthcare records of the group of patients into a testing subgroup, and a validating subgroup, wherein the electronic healthcare records of the testing subgroup are used to establish the predictive model.

4. The computer-implemented method of claim 3, further comprising:
validating the predictive model using the electronic healthcare records of the validating subgroup of patient; and
at least based on results of validating the predictive model, iteratively refining the predictive model.

5. The computer-implemented method of claim 1, wherein the features comprise: a preoperative factor, a demographic factor, and a perioperative factor, and wherein the elective surgical procedure comprises at least one of: a hip arthroplasty, and a knee arthroplasty.

6. The computer-implemented method of claim 1, wherein the machine learning algorithm comprises: a logistic regression (LR) algorithm, a linear discriminant analysis (LDA) algorithm, a Gaussian Naïve Bayes (GNB) algorithm, a random forest (RF) algorithm, an extreme Gradient Boosting (XGBoost) algorithm, a multilayer perceptron (MLP) algorithm, and an extra tress (ET) algorithm.

7. The computer-implemented method of claim 6, further comprising:
benchmarking the machine learning algorithm by at least one metric, wherein the at least one metric comprises: an area under receiver operating curve (AUROC), an area under precision-recall curve (AUPRC), an accuracy, a precision, a sensitivity, and a specificity.

8. A computer system comprising at least one hardware processor and at least one display, wherein the at least one hardware processor is configured to perform operations of:
accessing a database comprising electronic healthcare records of a group of patients, wherein each patient has received an elective surgical procedure;
based on the electronic healthcare records, extracting a data structure encoding a plurality of features of each patient from the group of patients, wherein a subset of the group of patients have received at least one hospital-based intervention after receiving the elective surgical procedure;
determining, using a machine learning algorithm that operates on the data structure, a Shapley value for each of the plurality of features, wherein the Shapley value indicates a likelihood for each patient with a corresponding feature to receive at least one hospital-based intervention;
identifying a subset of the plurality of features with absolute Shapley values higher than those of remaining features of the plurality of features; and
based on the subset of features, establishing a predictive model such that when electronic healthcare records of a new patient are received by the database, the predictive model predicts, based on the Shapley values for the identified subset of features, a combined likelihood for the new patient to receive a hospital-based intervention, wherein the combined likelihood is presented on the display.

9. The computer system of claim 8, wherein establishing the predictive model comprises:
summing the Shapley values of the identified subset of features; and
stratifying the summed Shapley values into a plurality of bins corresponding to respective levels of risk.

10. The computer system of claim 8, wherein the operations further comprise:
splitting the electronic healthcare records of the group of patients into a testing subgroup, and a validating subgroup, wherein the electronic healthcare records of the testing subgroup are used to establish the predictive model.

11. The computer system of claim 10, further comprising:
validating the predictive model using the electronic healthcare records of the validating subgroup of patient; and
at least based on results of validating the predictive model, iteratively refining the predictive model.

12. The computer system of claim 8, wherein the features comprise: a preoperative factor, a demographic factor, and a perioperative factor, and wherein the elective surgical procedure comprises at least one of: a hip arthroplasty, and a knee arthroplasty.

13. The computer system of claim 8, wherein the machine learning algorithm comprises: a logistic regression (LR) algorithm, a linear discriminant analysis (LDA) algorithm, a Gaussian Naïve Bayes (GNB) algorithm, a random forest (RF) algorithm, an eXtreme Gradient Boosting (XGBoost) algorithm, a multilayer perceptron (MLP) algorithm, and an extra tress (ET) algorithm.

14. The computer system of claim 13, wherein the operations further comprise:
benchmarking the machine learning algorithm by at least one metric, wherein the at least one metric comprises: an area under receiver operating curve (AUROC), an area under precision-recall curve (AUPRC), an accuracy, a precision, a sensitivity, and a specificity.

15. A non-transitory computer-readable medium comprising software instructions that, when executed by one or more computer processors, cause the one or more computer processors to perform operations of:
accessing a database comprising electronic healthcare records of a group of patients, wherein each patient has received an elective surgical procedure;
based on the electronic healthcare records, extracting a data structure encoding a plurality of features of each patient from the group of patients, wherein a subset of the group of patients have received at least one hospital-based intervention after receiving the elective surgical procedure;
determining, using a machine learning algorithm that operates on the data structure, a SHAP value for each of the plurality of features, wherein the Shapley value indicates a likelihood for each patient with a corresponding feature to receive at least one hospital-based intervention;
identifying a subset of the plurality of features with absolute Shapley values higher than those of remaining features of the plurality of features; and
based on the subset of features, establishing a predictive model such that when electronic healthcare records of a new patient are received by the database, the predictive model predicts, based on the Shapley values for the identified subset of features, a combined likelihood for the new patient to receive a hospital-based intervention.

16. The non-transitory computer-readable medium of claim 15, wherein establishing the predictive model comprises:
summing the Shapley values of the identified subset of features; and
stratifying the summed Shapley values into a plurality of bins corresponding to respective levels of risk.

17. The non-transitory computer-readable medium of claim 15, wherein the operations further comprise:
splitting the electronic healthcare records of the group of patients into a testing subgroup, and a validating subgroup, wherein the electronic healthcare records of the testing subgroup are used to establish the predictive model.

18. The non-transitory computer-readable medium of claim 17, wherein the operations further comprise:
validating the predictive model using the electronic healthcare records of the validating subgroup of patient; and
at least based on results of validating the predictive model, iteratively refining the predictive model.

19. The non-transitory computer-readable medium of claim 15, wherein the features comprise: a preoperative factor, a demographic factor, and a perioperative factor, and wherein the elective surgical procedure comprises at least one of: a hip arthroplasty, and a knee arthroplasty.

20. The non-transitory computer-readable medium of claim 15, wherein the machine learning algorithm comprises: a logistic regression (LR) algorithm, a linear discriminant analysis (LDA) algorithm, a Gaussian Naïve Bayes (GNB) algorithm, a random forest (RF) algorithm, an extreme Gradient Boosting (XGBoost) algorithm, a multilayer perceptron (MLP) algorithm, and an extra tress (ET) algorithm,
wherein the operations further comprise: benchmarking the machine learning algorithm by at least one metric, wherein the at least one metric comprises: an area under receiver operating curve (AUROC), an area under precision-recall curve (AUPRC), an accuracy, a precision, a sensitivity, and a specificity.
